(19) United States
(12) Patent Application Publication
(10) Pub. No.: US 2024/0277853 A1
(43) Pub. Date: Aug. 22, 2024

(54) MEDIUM-MOLECULAR-WEIGHT COMPOUND CONJUGATE FOR IMPROVING PHARMACOKINETIC PROFILE OF MEDIUM-MOLECULAR-WEIGHT COMPOUND AND METHOD OF PRODUCING THE SAME

(71) Applicants: TAGCYK Biotechnologies Inc., Tokyo (JP); The University of Tokyo, Tokyo (JP)

(72) Inventors: Susumu MUTO, Tokyo (JP); Shinsuke SANDO, Tokyo (JP); Miyuki HORI, Tokyo (JP); Haishun PIAO, Tokyo (JP); Kensuke OWARI, Tokyo (JP); Kazunobu FUTAMI, Tokyo (JP); Ryosuke UEKI, Tokyo (JP); Yutaro SAITO, Tokyo (JP); Seojung CHO, Tokyo (JP)

(73) Assignees: TAGCYX Biotechnologies Inc., Tokyo (JP); The University of Tokyo, Tokyo (JP)

(21) Appl. No.: 18/566,971

(22) PCT Filed: Jun. 3, 2022
(86) PCT No.: PCT/JP2022/022601

§ 371 Date: Dec. 4, 2023

Related U.S. Application Data
(60) Provisional application No. 63/196,927, filed on Jun. 4, 2021.

(51) Int. Cl. A61K 47/58; A61K 47/22; C12N 15/115

(52) U.S. Cl. CPC A61K 47/58; A61K 47/22; C12N 15/115

(57) ABSTRACT

Provided is a medium-molecular-weight compound conjugate capable of improving pharmacokinetic profile of a medium-molecular-weight compound, that is easy to obtain, safer to a biological body, and useful in vivo such as a nucleic acid aptamer, and a method of producing the same. The PMPC-medium-molecular-weight compound conjugate of the present invention is obtained by conjugating PMPC (poly-2-(methacryloyloxy)ethyl phosphoryicholine) to a medium-molecular-weight compound.

### TECHNICAL FIELD

The present invention relates to a medium-molecular-weight compound conjugate for improving pharmacokinetic profile of a medium-molecular-weight compound, and a method of producing the same.

### BACKGROUND ART

Nucleic acid aptamers are a single-stranded DNA or RNA molecule that can specifically bind to a target molecule. Nucleic acid aptamers are also called “artificial antibody”, or “chemical antibody” because these aptamers specifically bind to a target molecule by forming a thermodynamically stable conformation instead of a genetically controlled mechanism unlike other nucleic acid therapeutics, thereby expressing functions such as drug efficacy. Nucleic acid aptamers have target selectivity and affinity that often outstrip antibodies and can be produced simply by chemical synthesis unlike antibodies, which are proteins. For this reason, nucleic acid aptamers have been drawing attentions in recent years as a novel modality that has similar functions to antibodies and can be produced at low cost (NPL 1, NPL 2).

### CITATION LIST

### Non Patent Literature

### [NPL 1]

T. Adachi, Y. Nakamura, Molecules 2019, 24, 4229.

### [NPL 2]

Y. Zhang, B. C. Lai, M. Juhas Molecules 2019, 24, 941.

### SUMMARY OF INVENTION

### Technical Problem

Nucleic acid aptamers can have a variety of applications by selecting proteins to target like antibodies. On the other hand, nucleic acid aptamers have an extremely shorter blood half-life than antibodies. Thus, in most cases, such a short blood half-life stands in the way of developing nucleic acid aptamer therapeutic drugs for systemic administration method. Various methods to extend blood half-life of nucleic acid aptamers have been attempted. The leading cause of a short blood half-life of nucleic acid aptamers is considered progressing rapid renal excretion thereof, and, in a case of mouse, such aptamers are excreted in the urine in about several to 15 minutes after administration (A. D. Keefe, S. Pai, A. Ellington Nature Reviews Drug Discovery 2010, vol. 9, 537-550. (hereinafter referred to as “Literature 3”); J. Zhou, J. Rossi Nature Reviews Drug Discovery 2017, vol. 16, 181-202. (hereinafter referred to as “Literature 4”). Thus, the control of renal excretion rate of a nucleic acid aptamer directly links to the control of pharmacokinetic profile of the nucleic acid aptamer.

Various methods have been attempted to suppress the renal excretion of a nucleic acid aptamer. Examples of the method of extending blood half-life of a nucleic acid aptamer include a method of increasing a molecular weight, and a method of increasing lipophilicity.

An example of the method of increasing a molecular weight is a method of modifying an aptamer with a high-molecular-weight PEG (K. D. Kovacevic, J. C. Gilbert, B. Jilma Advanced Drug Delivery Reviews 2018, 134, 36-50, hereinafter referred to as “Literature 5”).

However, production of an anti-PEG antibody has been clinically confirmed in the PEG modification (A. Moreno, G. A. Pitoc, et al. Cell Chemical Biology, 2019, 26, 634-644. (hereinafter referred to as “Literature 6”); P. Zhang, F. Sun, S. Jiang Journal of Controlled Release 2016, 244, 184-193. hereinafter referred to as “Literature 7”). When the anti-PEG antibody is produced, it causes the activity loss of a nucleic acid aptamer and even the risk of anaphylactic shock by additional administration.

Examples of the method of increasing lipophilicity include a method of modifying using lipid soluble residue, and a method of increasing lipophilicity of the molecule itself. Examples of the method of modifying using lipid soluble residue include a method of modifying a base constituting a nucleic acid aptamer (S. Gupta, D. W. Drolet, et al. Nucleic Acid Therapeutics 2017 vol. 27, No. 6, 345-354, hereinafter referred to as “Literature 8”) and the like.

Examples of the method of increasing lipophilicity of the molecule itself include a method of modifying the phosphoric acid ester moiety by thiophosphorylation (S. Ni, H. Yao, et al. Int. J. Mol. Sci. 2017, 18, 1683, hereinafter referred to as “Literature 9”) and the like. However, while the lipophilicity of the obtained molecule is enhanced, safety issues have been revealed such as expression of toxicity that has not been recognized in the case of an unmodified aptamer (Journal of Nucleic Acids Therapeutics Society of Japan, 2020, 35-43.; W. Shen, C. C. DeHoyos, et al., Nucleic Acids Research, 2018, vol. 46(5), p. 2204-2217.). Use of a nucleic acid aptamer safely for a long term is desirable, but there has been no finding of such an aptamer that can be available by an easy method or a method of controlling the renal excretion of a nucleic acid aptamer that is safe to a biological body.

The present invention aims to provide a medium-molecular-weight compound conjugate capable of improving pharmacokinetic profile of a medium-molecular-weight compound useful in vivo such as a nucleic acid aptamer, and that is easy to obtain, highly safe to a biological body, and administerable for a long term, and a method of producing the same.

### Solution to Problem

The first aspect of the present invention is a PMPC-medium-molecular-weight compound conjugate for improving pharmacokinetic profile of a medium-molecular-weight compound, wherein PMPC (poly-2-(methacryloyloxy)ethyl phosphorylcholine) is conjugated to the medium-molecular-weight compound.

PMPC is a highly water-soluble bipolar polymer known to have extremely high biocompatibility because of having the same structure as the lipid constituting the cell membrane. No published reports on toxicity, antibody production and the like originated from PMPC has been known.

In the first aspect, the medium-molecular-weight compound may be a single-stranded or double-stranded oligonucleotide having a molecular weight of 50 kDa or less.

In the first aspect, the oligonucleotide may be a DNA aptamer or an RNA aptamer.

In the first aspect, the medium-molecular-weight compound may be a peptide or a protein having a molecular weight of 50 kDa or less.

In the first aspect, the medium-molecular-weight compound may be a nanobody, a Fab fragment of an antibody, a peptide hormone, a chemokine, a cytokine, a linear or cyclic peptide having a function to bind to a specific protein.

In the first aspect, PMPC to be conjugated may have a degree of polymerization n of n=50 to 800.

The second aspect of the present invention is a method of producing a PMPC-medium-molecular-weight compound conjugate comprising a polymerization step of subjecting MPC to polymerization reaction using an initiator to obtain PMPC having a protected or unprotected terminal primary amino group, and a binding step of chemically binding a medium-molecular-weight compound and PMPC using the terminal primary amine of PMPC.

In the second aspect, the production method may further comprise, when PMPC with a protected terminal primary amino group is obtained in the polymerization step, a deprotecting step of removing a protecting group of the terminal primary amino group of PMPC after the polymerization step.

In the second aspect, the initiator in the polymerization step to obtain PMPC is 2-(tert-butyloxycarbonyl-aminoethyl) isobutylbromide, and deprotection reaction that removes Boc protecting group of the terminal primary amino group of PMPC may be performed following the polymerization reaction.

In the second aspect, the medium-molecular-weight compound may be a single-stranded or double-stranded oligonucleotide having a molecular weight of 50 kDa or less.

In the second aspect, the oligonucleotide may be a DNA aptamer or an RNA aptamer.

In the second aspect, the medium-molecular-weight compound may be a peptide or a protein having a molecular weight of 50 kDa or less.

In the second aspect, the medium-molecular-weight compound may be a nanobody, a Fab fragment of an antibody, a peptide hormone, a linear or cyclic peptide having a function to bind to a specific protein.

In the second aspect, the medium-molecular-weight compound and PMPC may be chemically bound through a linker.

In the second aspect, the linker and PMPC may be bound by condensation reaction of an active ester and an amine, or condensation reaction of an amine and an intermediate formed by activating an ester or a carboxylic acid in the reaction system or outside the reaction system.

In the second aspect, the active ester may be N-hydroxysuccinimide ester.

In the second aspect, the DNA aptamer or the RNA aptamer has a functional group used for click reaction, and the functional group of the DNA aptamer or the RNA aptamer and PMPC may be bound by click reaction.

### Advantageous Effects of Invention

In the present invention, a conjugate to which PMPC is bound is made for increasing a molecular weight of the medium-molecular-weight compound such as a nucleic acid aptamer. The PMPC conjugate is highly biocompatible and rarely causes the antibody production as found in the conventionally used PEG conjugates. For this reason, such a conjugate can be a safe molecule to a biological body to which the conjugate is administered, thereby enabling long-term drug administration. The medium-molecular-weight compound useful in vivo such as a nucleic acid aptamer, when made into a conjugate with PMPC, can extend blood half-life of the medium-molecular-weight compound useful in vivo and improve pharmacokinetic profile.

### BRIEF DESCRIPTION OF DRAWINGS

FIG. 1 is a chart showing experimental results that confirmed half-life in blood and AUC of PMPC-aptamer conjugates with four different degrees of MPC polymerization, a PEG-aptamer conjugate, and an aptamer only.

FIG. 2A is a chart showing experimental results that measured IFN γ signaling inhibitory activity using an aptamer only.

FIG. 2B is a chart showing experimental results that measured IFN γ signaling inhibitory activity using PMPC-aptamer conjugates.

FIG. 2C is a chart showing experimental results that measured IFN γ signaling inhibitory activity using a PEG-aptamer conjugate.

### DESCRIPTION OF EMBODIMENTS

Hereinafter, an embodiment of the conjugate for improving pharmacokinetic profile of a medium-molecular-weight compound according to the present invention, and the method of producing the same will be described.

Waste products from the blood are filtered through the renal glomerulus and excreted in the urine. Highly water-soluble molecules having a molecular weight of 50000 (50 kDa) or less are considered to be easily excreted. Nucleic acid aptamers have an average molecular weight of about 10000 (10 kDa) to 15000 (15 kDa) and are extremely water-soluble, thereby having the property of being extremely easily excreted from the kidneys. For improving such an easy excretion, various attempts have been made to delay the renal excretion as mentioned earlier. As a result, it is shown that the blood half-life can be extended by making a nucleic acid aptamer into a lipophilic molecule, making a nucleic acid aptamer into a macromolecule, or a combination method of both (Literatures 3, 4, 5, 8, and 9).

However, making a nucleic acid aptamer into a lipohilic molecule has shown some issues such as soaring production costs of a nucleic acid aptamer due to chemical modification to a base or a sugar in the nucleic acid aptamer, and expression of toxicity that has not been recognized for an unmodified aptamer when a sulfur atom is introduced to phosphoric acid ester bond. Making a nucleic acid aptamer into a macromolecule shows notable improvement of pharmacokinetic profile of a nucleic acid aptamer when the nucleic acid aptamer is modified with PEG having an average molecular weight of 40000 or more. Making a nucleic acid aptamer into a macromolecule has been increasingly used as a comparatively inexpensive effective modification method for improving pharmacokinetic profile of nucleic acid aptamers as seen in commercialization of pegaptanib (Macugen (registered trademark)) by Gilead Sciences, Inc., starting of the clinical research of ARC1779 by Archemix Corp and the like. However, production of the anti-PEG antibody has been detected in the clinical use of the PEG-modified nucleic acid aptamer (Literatures 6 and 7). Under such circumstances, not only the activity loss of the nucleic acid aptamer but also the risk of anaphylactic shock by production of the anti-PEG antibody is caused. For this reason, the improvement of pharmacokinetic profile by making a nucleic acid aptamer into a macromolecule needed urgent development of a novel polymer for modification in place of PEG for practical use of nucleic acid aptamers.

The present inventors conducted studies using poly-MPC (PMPC), a polymer of 2-(methacryloyloxy)ethyl phosphorylcholine (MPC), as a polymer for modification to make a medium-molecular-weight compound such as nucleic acid aptamers into a macromolecule.

PMPC has the phosphorylcholine structure similar to the phospholipid forming cell membrane, in other words, a bipolar neutral polymer having both positive charge and negative charge. PMPC is known to have extremely high biocompatibility due to its structure that mimics the cell membrane surface, and thus has already been practically used as a coating agent for artificial joint, artificial organ, artificial blood vessel and the like. Evidently, PMPC is a highly safe polymer with no report on antigenicity at present (Masayuki Kyomoto, Artificial organs, 2015, vol. 44 No. 3 p. 161-163.; Takayuki Kido, Chisato Nojiri et al., Artificial organs, 1999, vol. 28 No. 1 p. 196-199.; K. Ishihara, Y. Goto, et al. Biochimica et Biophysica Acta 2011, 1810, 268-275.). PMPC has the structure that mimics the phospholipid of the cell membrane outer surface present in a large amount in vivo, and is thus less likely to generate the antibody thereof. In respect of the production, NOF Corporation, a Japanese company, succeeded in mass production and has been selling PMPC, thereby PMPC is a practical polymer easily obtainable due to the already established production method.

The present inventors produced a conjugate of a nucleic acid aptamer and PMPC by subjecting MPC to polymerization reaction using an initiator having the terminal N-Boc (tert-butoxycarbamate) structure, performing deprotection reaction that removes the protecting group of the terminal amino group after completion of the polymerization reaction, and chemically binding PMPC to an aptamer using the produced primary amine. Additionally, using the produced conjugate, blood half-life of the conjugate in the body of animals was evaluated comparatively. The results (details to be described later) revealed that the blood half-life of the conjugate wherein PMPC is bound to the aptamer demonstrated about the same length of time as the blood half-life of the conjugate wherein PEG and an aptamer are bound and can commonly be used as a means to suppress renal excretion, thereby showing that the conjugation with PMPC is a practical method. The conjugate of PEG and an aptamer generally decreases the affinity to a target of the aptamer chemically binding to PEG, whereas the conjugate of PMPC and an aptamer, as shown in Example 5 to be described later, did not at all decrease the affinity to a target of the aptamer. Thus, the present inventors have found that the PMPC conjugation is superior modification to the conventionally used PEG conjugation in respect of the blood half-life and the affinity to a target of the aptamer, thereby the present invention has been accomplished.

The PMPC-medium-molecular-weight compound conjugate of the present invention is the molecule capable of reducing the renal excretion rate or suppressing the renal excretion of a medium-molecular-weight compound due to the physically enlarged molecule. Applying this principle, an oligonucleotide can be used as an example of the medium-molecular-weight compound to be the target to which PMPC is conjugated. The oligonucleotide may be a DNA aptamer or an RNA aptamer. In the present description, the DNA aptamer and the RNA aptamer may be expressed as the “nucleic acid aptamer.”

Another example of the medium-molecular-weight compound to be the target to which PMPC is conjugated include highly water-soluble peptides with low protein binding ability, or proteins having a molecular weight of 50 kDa or less. Of such peptides or proteins, highly water-soluble compounds whose major excretion pathway is renal excretion are used further effectively. Specifically, a peptide hormone, a nanobody, a Fab fragment of an antibody, a chemokine, a cytokine, a linear or cyclic peptide having a function to bind to a specific protein can be used as the medium-molecular-weight compound.

The chemically binding site of a nucleic acid aptamer to PMPC can be any location as long as the activity is not affected, as well as the nucleobase moiety located at the 5′- or 3′-end of a generally used nucleic acid aptamer, and the 5′- or 3′-terminal hydroxyl group. For example, —N₃ (azide group), —NH₂ (amino group), —COOH (carboxyl group), —CONH₂ (amide group), —OH (hydroxyl group), —SH (thiol group), —CC— (alkynyl group), —CHO (formyl group), —CO— (carbonyl group), —CHCH₂ (vinyl group), —NH—NH₂ (hydrazide group), or a N-substituted maleimide group is introduced to a nucleobase moiety or a linker moiety other than the 5′- or 3′-terminal of a nucleic acid aptamer, thereby chemically binding to PMPC using these functional groups.

As is the case in the nucleic acid aptamer, the PMPC modification can also be achieved at any moiety irrelevant to the activities of these in a molecule of a peptide or a protein.

The medium-molecular-weight compound as described above, when formed as a conjugate with PMPC, can improve the blood half-life thereof.

Examples of the “medium-molecular-weight compound” applicable in the present invention include, as well as the above listed oligonucleotides and peptides or proteins having a molecular weight of 50 kDa or less, oligonucleotides having a molecular weight of 1 kDa to 50 kDa, biopolymers other than peptides or proteins, and chemically synthesized non-natural compounds having a molecular weight of 1 kDa or more and 50 kDa or less.

The “pharmacokinetic profile” in the present description refers to the behavior expressed by the parameter calculated from temporal concentration changes in blood of an administered medium-molecular-weight compound such as an aptamer, a peptide, or a protein, and is evaluated using parameters such as blood half-life (T1/2), Volume of distribution (Vd), Area Under the blood concentration-time Curve (AUC), maximum drug concentration (Cmax), Time to maximum drug concentration (Tmax) as indicators. The “improving pharmacokinetic profile” in the present description refers to, for example, extended T1/2 and increased AUC and the like of the medium-molecular-weight compound administered to blood. Specific example of the “state where pharmacokinetic profile is improved” refers to the state where an appropriate blood concentration and a blood exposure dose can be maintained so that the administered medium-molecular-weight compound functions as a drug, such as T1/2 of an aptamer administered to blood is controlled to delay renal excretion thereof.

The “making into a macromolecule” in the present description means to increase a molecular weight by chemically binding a polymer to a medium-molecular-weight compound. More specifically, a polymer having a molecular weight of 5 kDa to 80 kDa is bound to a medium-molecular-weight compound having a molecular weight of 1 kDa to 50 kDa to produce a conjugate having a molecular weight of 6 kDa to 130 kDa.

### Method of Linking

For chemically binding the medium-molecular-weight compound and PMPC, a linker of any length that mediates the medium-molecular-weight compound and PMPC may be used. For the linker structure that can be used includes a linear or branched carbon chain whose substituted or unsubstituted main skeleton is constituted only of carbon atoms, a linear or branched carbon chain, a linear or branched carbon chain whose main skeleton includes a heteroatom other than carbon atom, a peptide chain, short-chain PEG composed of 30 or less PEG units (“1 unit” is a link of 4 atoms) and long-chain PEG having a longer chain length than short-chain PEG, and an oligonucleotide and the like. The linker structure is not limited to the structures constituted of a single kind of molecule structure, and several of the above structures can be used in combination such as a structure wherein a carbon chain and a peptide chain are bound.

The chemically binding reaction of the medium-molecular-weight compound and PMPC can be achieved by condensation reaction of an active ester and an amine, or condensation reaction of an amine and an intermediate formed by activating an ester or a carboxylic acid in the reaction system or outside the reaction system, but is not limited thereto.

For the condensation reaction of an active ester and an amine, an active ester represented by NHS ((N-hydroxysuccinimide)ester) can be used. Further, pre-activated compounds such as acid halide, acid anhydride, and acid azide can be used as an activated form of a carboxyl group and the like.

The reagent for activating an ester or a carboxylic acid in the reaction system or outside the system can be a dehydration condensation agent such as carbodiimide, BOP reagent, DMT-MM (4-(4,6-dimethoxy-1,3,5-triazin-2-yl)-4-methylmorpholinium Chloride), HOBT (1-hydroxybenzotriazole), and HOAT (1-hydroxy-7-azobenzotriazole).

For the condensation reaction, click reaction, production reactions of thioether, ether and ester can also be used. The reactions described above can be used singly or in combination.

A molecular compound has a functional group used for click reaction, and the functional group and PMPC are bound by click reaction. Click reaction is not limited to the condition of adding a copper catalyst, and can be carried out under the reaction of not adding a copper catalyst depending on a functional group to be a reactive site of a substrate to be used. Examples of the functional group used for click reaction include an azido group, a linear alkynyl group, a cyclic alkynyl group, but are not limited thereto.

The functional group to be the reactive site to the linker in a PMPC molecule is included in the terminal moiety of the PMPC molecule after polymerization. Such a functional group to be the reactive site is included in the structure of the compound to be the initiator of the MPC polymerization to obtain the PMPC molecule. Usable active functional groups of the compound to be the initiator are —NH₂ (amino group), —COOH (carboxyl group), —CONH₂ (amide group), —OH (hydroxyl group), —SH (thiol group), —N; (azide group), —CC— (alkynyl group), —CHO (formyl group), —CO— (carbonyl group), —CHCH₂ (vinyl group), —NH—NH₂ (hydrazide group), a N-substituted maleimide group and the like. Of these functional groups, those require a protecting group during the polymerization reaction of MPC can be protected as needed with a removable protecting group that is stable under the polymerization reaction conditions of MPC and does not affect the structure of the produced PMPC. The protecting group may be N-Boc, but is not limited thereto. The polymerization can be achieved with the terminal primary amine of the initiator as being unprotected depending on conditions of the polymerization reaction of MPC, and in such a case PMPC with the unprotected terminal primary amine is obtained. Such an amino group can also be used for the subsequent chemically binding reaction of PMPC and the medium-molecular-weight compound without introducing a protecting group thereto.

In the present description, the method of linking using click reaction will be described as an example in the example to be described later. In the reaction using NHS active ester as versatile as click reaction, PMPC having a terminal carboxyl group is produced using a known method and used (H. Han. S. Zhang, et al. Polymer, 2016, 82, 255-261; B. Yu. A. Lowe, K. Ishihara Biomacromolecules 2009, 10,950-958; D. Miyamoto, J. Watanabe, K. Ishihara Biomaterials 2004, 25, 71-76.) A polymerization example that uses an initiator with a terminal NHS ester is also known, and PMPC activated with the NHS ester can be produced by using such a polymerization method (A. Lewis, Y. Tang, et al. Bioconjugate Chem. 2008, 19, 2144-2155.)

The PMPC having a terminal carboxyl group is activated using an activating reagent such as carbodiimide, BOP reagent, DMT-MM, HOBT, HOAT and the like and reacted to N-hydroxysuccinimide to produce NHS ester of PMPC. The produced NHS ester can be bound by the method as shown in Step 2 of Example 2 to the oligonucleotide having the terminal primary amine produced by the method as shown in Step 1 of Example 2 to be described later.

The terminal NHS ester of PMPC, when simply mixed with a peptide or a protein in buffer, can also chemically bind the lysine residue in the peptide or the protein to PMPC.

The molecular weight of PMPC used in the conjugation can be adjusted by controlling the degree of polymerization with different polymerization reaction conditions.

The conjugate can control the blood half-life by adjusting the molecular weight of PMPC used for the modification, and the larger a molecular weight, the longer T1/2 due to inhibited renal excretion.

Usable PMPC to be conjugated have a degree of polymerization n of n=50 to 800.

Use of a branched linker as the linker molecule enables PMPC to chemically bind in the range from 1 to 5, and preferably 1 to 3. Examples of the branched linker include a peptide chain having, in a chain, one kind of amino acid such as aspartic acid (carboxyl group), glutamic acid (carboxyl group), cysteine (thiol group), and lysine (amino group), or having two to three kinds of amino acids introduced as needed in accordance with the binding method. These peptides can change binding modes for each of the functional groups at reactive sites, and a payload such as polymers, low molecular drugs and the like of different kinds can be bound to the medium-molecular-weight compound through linkers of the same kind of skeleton or different kinds of skeleton having functional groups reactable to each of the functional groups, respectively. The amino acid to be used can be artificially synthesized amino acids as well as the naturally occurring amino acids.

The further extension of blood half-life can be expected when PMPC having a large molecular weight (about 5 kDa to 80 kDa) is bound because the contact between a nucleolytic enzyme and an aptamer is inhibited.

Also in the case of the medium-molecular-weight compound being a peptide, PMPC having a large molecular weight (about 5 kDa to 80 kDa) bound thereto inhibits the contact to a protease and thereby inhibits the decomposition in blood, thereby further extension of blood half-life can be expected.

The PMPC-medium-molecular-weight compound conjugate of the present invention can be used as a test reagent, an active pharmaceutical ingredient, a drug for human, and a drug for animals, singly or in combination with other drug additives. Further, the present conjugate is applicable to clinical instrument including reagents for diagnosis when combined with other medical materials such as artificial bone, artificial blood vessel, artificial heart, stent, medical tube, connector, and medical pump.

### EXAMPLES

### [Example 1] Production of PMPC with DBCO (DBCO-PMPC) for Conjugation

### Step 1 Synthesis of 2-((tert-butoxycarbonyl)amino)ethyl 2-bromo-2-methylpropanoate (Initiator)

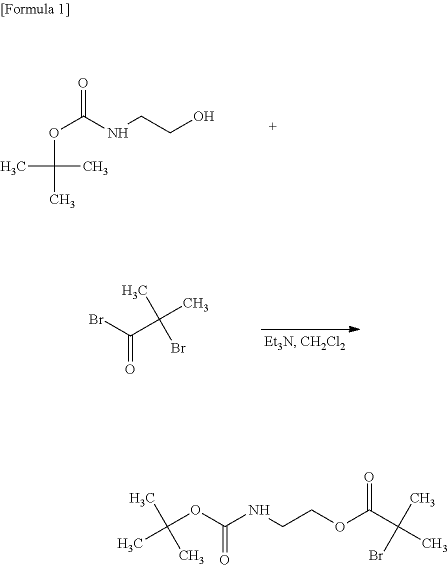

Triethylamine (1.6 mL, 11.5 mmol) and N-(tert-butoxycarbonyl) ethyl alcohol (1.62 g, 10.0 mmol) were dissolved in dehydrated dichloromethane (10 mL), and 2-bromo-propanoyl bromide (1.28 mL, 10.4 mmol) was slowly added dropwise while stirring on ice bath. After stirring for 4 hours at room temperature, the reaction solution was filtered and distilled under reduced pressure. The obtained residue was column-purified (developing solvent: hexane/diethyl ether=8/2, v/v), thereby obtaining the title compound (2.24 g, 72%).

¹H NMR (400 MHz, CDCl₃) δ 4.81 (1H, s), 4.24 (2H, t, J=5.0 Hz), 3.42-3.48 (2H, m), 1.95 (6H, s), 1.45 (9H, s).

### Step 2 Production of TFA Salts of PMPC Having Terminal Primary Amine (NH₂-PMPC)

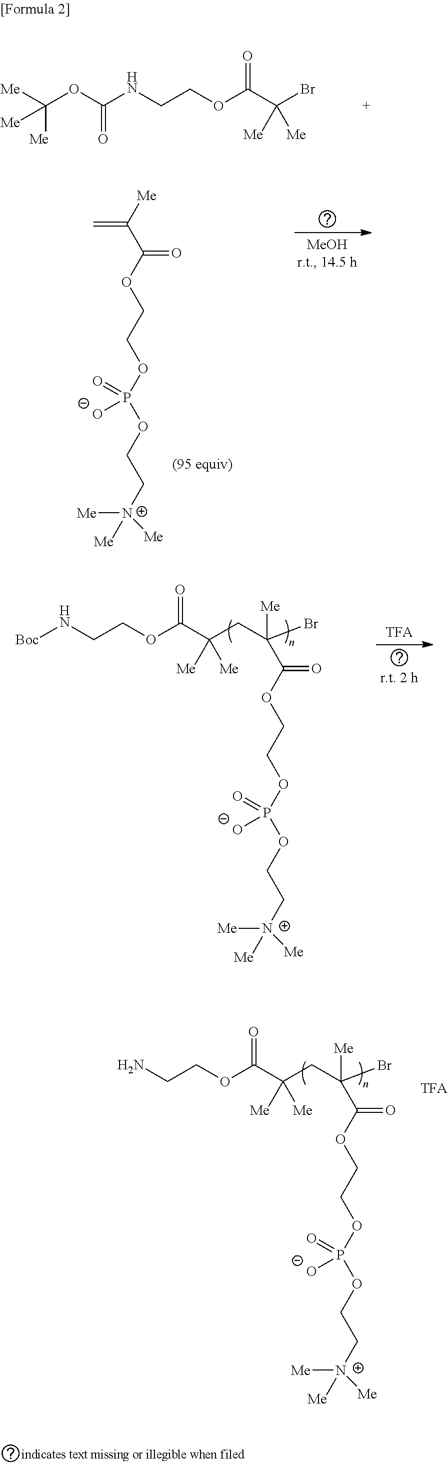

Copper(I) bromide (1.1 mg, 7.7 μmol), bipyridine (2.3 mg, 15 μmol), MPC (206 mg, 0.70 mmol) were added to 20 mL of degassed methanol, further the initiator (2.1 mg, 6.8 μmol) produced in Step 1 was added thereto, sealed, and stirred for 14.5 hours at room temperature.

After removing the solvent from the reaction solution by evaporation, 2 mL of TFA was added and stirred for 2 hours to remove Boc protecting group. The reaction solution was added dropwise to 40 mL of diethyl ether under ice-water cooling while stirring, and the formed solid was filtered, thereby obtaining a white solid. The obtained solid was dissolved in a small amount of water, 0.5 M EDTA was added thereto, the solution was dialyzed using a Regenerated Cellulose (RC) dialysis membrane, freeze dried, thereby obtaining the polymer having n=100 of the title compound as a white solid (yield 60 to 80).

In the above Step 2, the polymerization reactions were carried out using respectively 103 mg (0.35 mmol) of PMC in the case of a degree of polymerization n=50, 417 mg (1.41 mmol) of PMC in the case of a degree of polymerization n=200, 824 mg (2.79 mmol) of PMC in the case of a degree of polymerization n=400, thereby obtaining PMPC having n=50, n=200 and n=400 as white solids as in the case of n=100 described above (yield: 70 to 93%).

For each of the produced MPC polymers, the calculation of an intensity comparison between a signal derived from the polymer and a signal derived from the terminal t-butyl group by 1H-NMR measurement confirmed that the obtained polymers had the desired degrees of polymerization. The actual measured values of calculated degrees of polymerization are shown below.

| Estimated degrees of | Calculated values by NMR |
|---|---|
| polymerization | spectrum analysis |
| n = 50 | 46 |
| n = 100 | 88 |
| n = 200 | 220 |
| n = 400 | 347 |

### Step 3 Production of PMPC with DBCO (DBCO-PMPC) P24C3

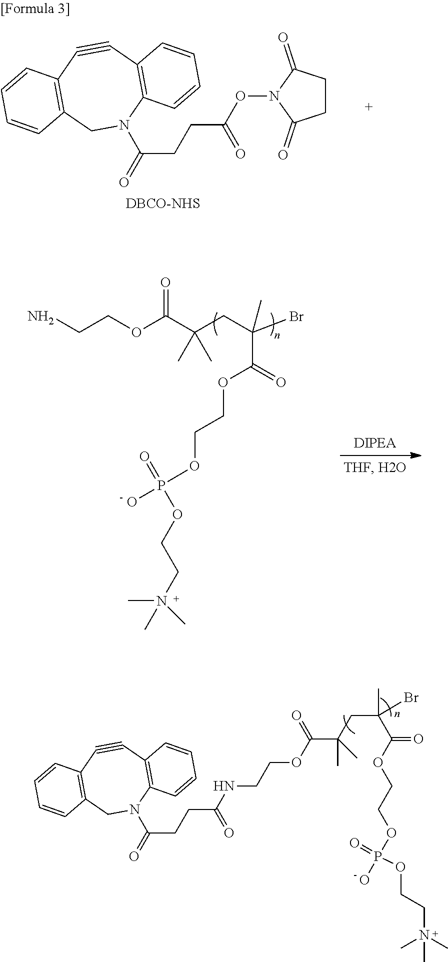

TFA salts of NH₂-PMPC produced in the above Step 2 and having four different degrees of polymerization (1 equivalent amount, 40 to 350 mg) were dissolved in a mixed solvent of water and THF, and diisopropylethylamine (3 equivalent amount) was added thereto. Commercial dibenzocyclooctyne-N-hydroxysuccinimidyl ester (DBCO-NHS ester) (2 equivalent amount) was added to the reaction solution and stirred for 24 hours at room temperature. After distilling the solvent of the reaction solution, water was added to filter out impurities, then the obtained filtrate was dialyzed using a Regenerated Cellulose (RC) dialysis membrane and freeze dried, thereby obtaining the title compound (yield: 50 to 80%).

### [Example 2] Production of Azide Adduct of Aptamer

### Step 1 Synthesis of DNA Aptamer

Using a commercial amino-modifier C6-dT Amidite, a DNA aptamer in which amino-modifier C6-dT was introduced into the X part of the following sequence, which is the sequence of an IFN γ-bound aptamer, was synthesized in accordance with the method described in PCT International Publication No. WO 2016/143700.

TABLE 1

| | |
|---|---|
| SEQ ID | 5′d(CCCGCCCGGGTCCGCGAAGCGGTAGGT-Ds- |
| NO: 1 | TGGGCTAGGC-Ds-GCTGGGGGGCGCGXAGCG)-3′ |

### Step 2 Production of Azide Adduct of Aptamer

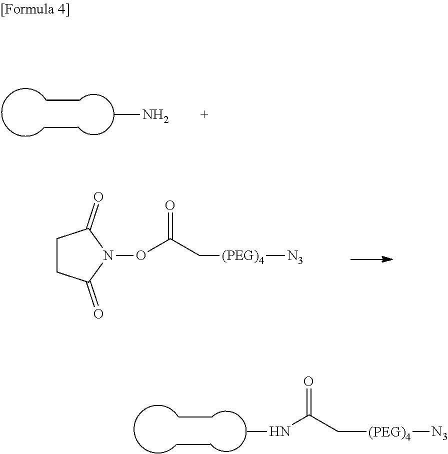

The DNA aptamer produced in Step 1 and in which a side chain having an amino group at the nucleobase moiety of the X part (dT) in the SEQ ID NO: 1 was added was dissolved in PBS buffer (100 μM), 3 to 5 equivalent amount of commercial N₃-PEG₄-NHS was added thereto and stirred for 24 hours at room temperature. The reaction solution was purified by gel filtration, thereby obtaining the azide adduct of DNA aptamer in a yield of 60%.

### [Example 3] Production of PMPC-Aptamer Conjugates

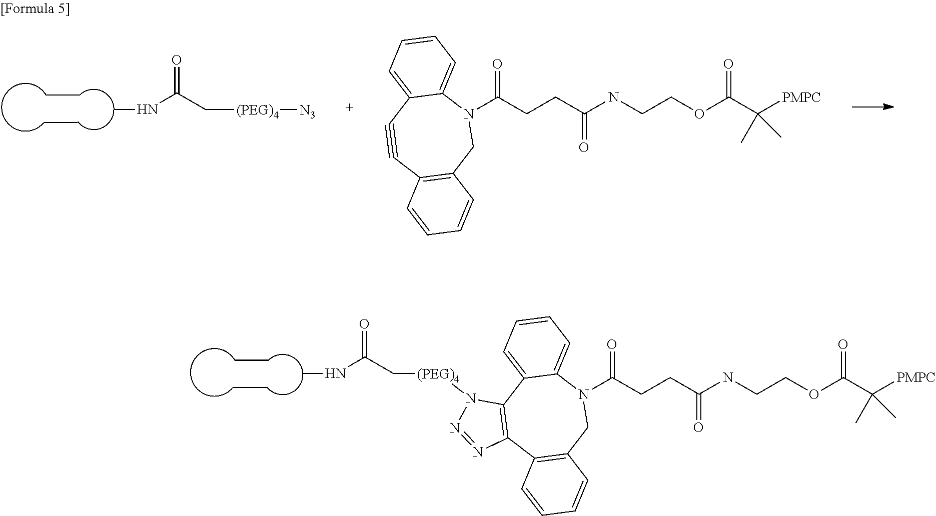

DBCO-PMPC (5 equivalent amount relative to the amount of aptamer) produced in Step 3 of Example 1 and having four different degrees of polymerization were dissolved in 50% DMSO (100 μM), and the azide adduct of DNA aptamer produced in Step 2 of Example 2 (1 equivalent amount) was added to the solutions respectively, and then stirred for 24 hours at room temperature. The reaction solution was purified by gel filtration to remove unreacted aptamer, thereby obtaining aptamer-PMPC conjugates having four different degrees of MPC polymerization as freeze dried conjugates in a yield of 60 to 70%.

### [Example 4] Study on the Pharmacokinetic Profile of PMPC-Aptamer Conjugates

The PMPC-aptamer conjugates produced in Example 3 and having four different degrees of MPC polymerization were intravenously administered respectively to four 6-week-old male rats (1 mg/kg in terms of aptamer amount), changes in blood concentration of the aptamer in each rat were measured by the qPCR method. For Comparative Example, an unmodified aptamer only to which PMPC was not conjugated and a conjugate of an aptamer and PEG having a molecular weight of 40000 (PEG (40000)-aptamer conjugate) were respectively administered in the same dose to rats, and changes in blood concentration of the aptamer in the rats were similarly measured.

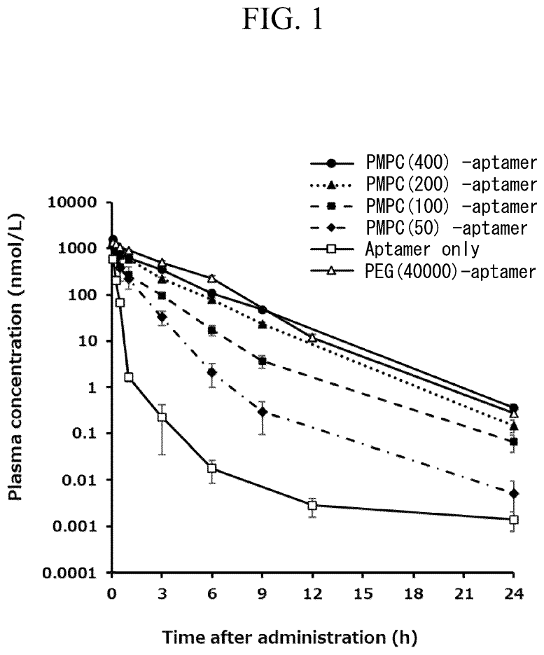

The results are shown in FIG. 1. In FIG. 1, for example, PMPC (400)-aptamer refers to the conjugate of PMPC having the degree of polymerization n of n=400 and an aptamer. In FIG. 1, the vertical line of the graph shows the aptamer concentration in plasma and the horizontal line shows the time after aptamer administration, respectively. The graph of FIG. 1 showed that the decrease of blood (in plasma) concentration within 3 hours after aptamer administration was slower in proportion to the molecular weight of conjugated PMPC, thereafter the decrease rate of aptamer amount became slower, and the retention time and retention amount in blood were apparently increased. These results indicate that when the PMPC-aptamer conjugates were administered, the half-life in blood extends in a PMPC-molecular-weight-dependent-manner and AUC (Area Under the blood concentration-time Curve) increases when compared to the case where the aptamer only is administered. This finding revealed the significant improvement of pharmacokinetic profile of the aptamer. In the comparison between the PEG (40000)-aptamer conjugate and the PMPC-aptamer conjugates, the PMPC (400)-aptamer and the PMPC (200)-aptamer demonstrated about the same pharmacokinetic profile to the aptamer-PEG (40000) conjugate. Considering such a result, the PMPC-aptamer conjugates demonstrate about the same effect to PEG-modified conjugates that have been conventionally used, and the PMPC modification is expected to be an alternative technology to the PEG modification.

### Example 5

The aptamer only (unmodified aptamer) produced in Step 1 of Example 2, the PMPC (400)-aptamer conjugate produced in Example 3, and the PEG (40000)-aptamer conjugate were respectively measured for IFN γ signaling inhibitory activity using MDA-MB-231 cells. IFN γ in 2 nM/mL and the above aptamer compounds were allowed to act to cultured cells to detect the phosphorylation of STAT1 by FCM (Flow cytometry). Amounts of the above aptamer compounds were changed from equimolar amount (1 eq) to 100 times molar amount (100 eq) relative to the amount of IFN γ.

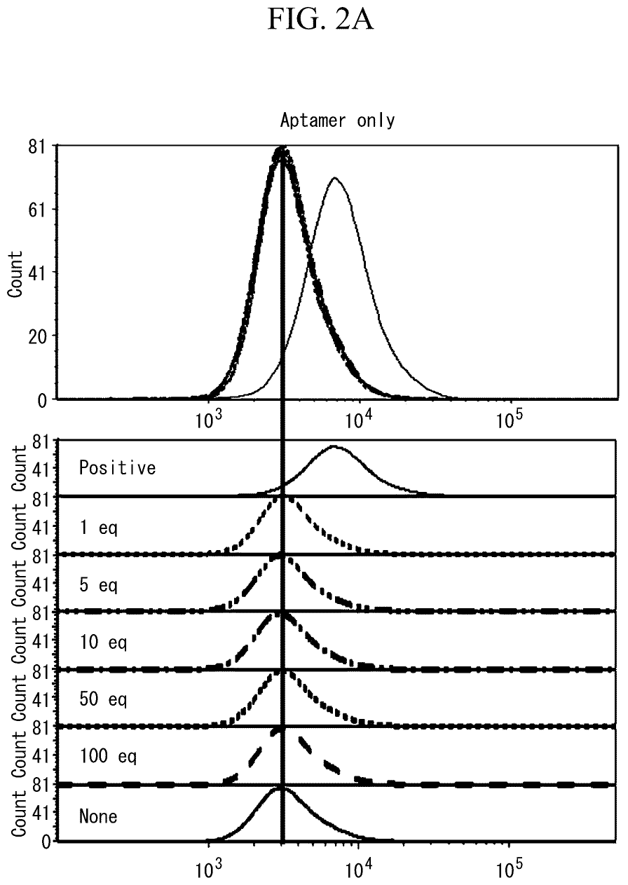

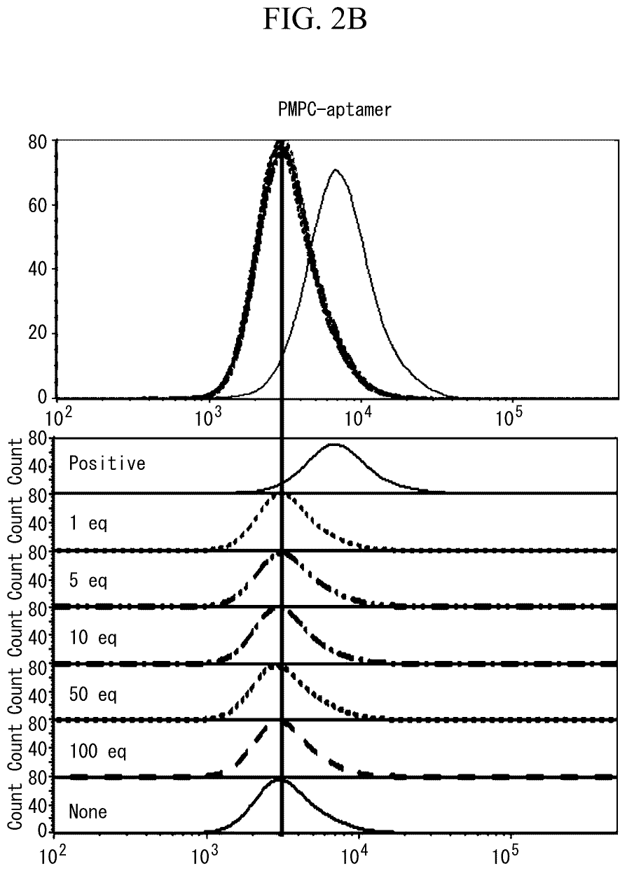

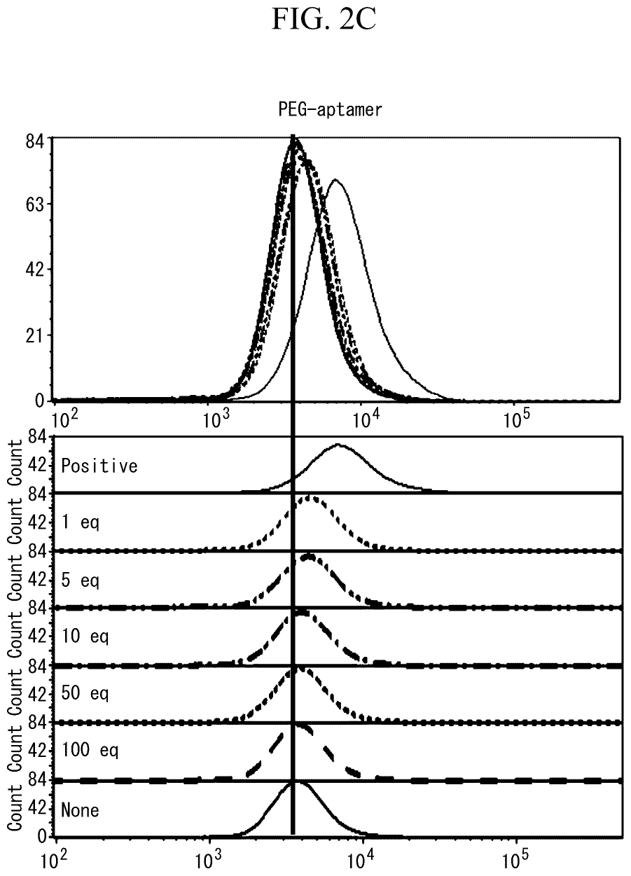

The results are shown in FIG. 2A to FIG. 2C. FIG. 2A shows the result obtained using the aptamer only, FIG. 2B shows the results obtained using the PMPC (400)-aptamer, and FIG. 2C shows the result obtained using the PEG (40000)-aptamer, respectively. The vertical line in each figure shows the aptamer concentration, and the horizontal line shows the peak shift amount by flow cytometry, respectively, and the graph above shows the overlaid results of each graph below. The “Positive” in FIG. 2A to FIG. 2C represents the amount of phosphorylated STAT1 when the IFN γ signal completely enters and the phosphorylation proceeds, and the “none” represents the amount of phosphorylated STAT1 in the case of unstimulated (background). In the case of the IFN γ activity being completely inhibited, the peak shift does not occur when compared against the background, whereas in the case where the IFN γ activity occurs, the peak shifts rightward in response to the activity intensity (right side from the vertical line in FIG. 2). In both cases of the aptamer only (FIG. 2A) and the PMPC-aptamer conjugates (FIG. 2B), the peak shifts by the phosphorylation were suppressed at 1 eq (equimolar amount) relative to the amount of IFN γ, thereby revealing substantially complete inhibition of the IFN γ phosphorylation (IFN γ activity) of STAT1. On the other hand, the PEG-aptamer (40000) conjugate (FIG. 2C) had the peak top shifted rightward even when 5 eq (5 times molar amount) was used, thereby revealing that the STAT1 phosphorylation was not completely inhibited and the aptamer activity decreased by the PEG modification. The above results proved that the PMPC addition to the aptamers is irrelevant to the aptamer activity.

For making a medium-molecular-weight compound such as a nucleic acid aptamer into a macromolecule, the present invention employs PMPC as a polymer that is alternative to PEG that has been conventionally used and less likely to produce an antibody, and forms the aptamer-PMPC conjugate. The modification of an aptamer with PMPC enables to obtain a safer conjugate while maintaining about the same renal excretion suppression effect as the PEG-modification that have been conventionally practiced. Accordingly, long-term drug administration is expected to be enabled. Additionally, the modification with PMPC can improve the pharmacokinetic profile of a medium-molecular-weight compound useful in vivo such as a nucleic acid aptamer.

1. A PMPC-medium-molecular-weight compound conjugate for improving pharmacokinetic profile of a medium-molecular-weight compound, wherein PMPC (poly-2-(methacryloyloxy)ethyl phosphorylcholine) is conjugated to the medium-molecular-weight compound.

2. The PMPC-medium-molecular-weight compound conjugate according to claim 1, wherein the medium-molecular-weight compound is a single-stranded or double-stranded oligonucleotide having a molecular weight of 50 kDa or less.

3. The PMPC-medium-molecular-weight compound conjugate according to claim 2, wherein the oligonucleotide is a DNA aptamer or an RNA aptamer.

4. The PMPC-medium-molecular-weight compound conjugate according to claim 1, wherein the medium-molecular-weight compound is a peptide or a protein having a molecular weight of 50 kDa or less.

5. The PMPC-medium-molecular-weight compound conjugate according to claim 1, wherein the medium-molecular-weight compound is a nanobody, a Fab fragment of an antibody, a peptide hormone, a chemokine, a cytokine, a linear or cyclic peptide having a function to bind to a specific protein.

6. The PMPC-medium-molecular-weight compound conjugate according to claim 1, wherein PMPC to be conjugated has a degree of polymerization n of n=50 to 800.

7. A method of producing a PMPC-medium-molecular-weight compound conjugate comprising:
a polymerization step of subjecting MPC to polymerization reaction using an initiator to obtain PMPC having a terminal primary amino group, and
a binding step of chemically binding a medium-molecular-weight compound and PMPC using the terminal primary amine of PMPC.

8. The method according to claim 7, further comprising, when PMPC with a protected terminal primary amino group is obtained in the polymerization step, a deprotecting step of removing a protecting group of the terminal primary amino group of PMPC after the polymerization step.

9. The method according to claim 7, wherein when the initiator in the polymerization step is 2-(tert-butyloxycarbonyl-aminoethyl) isobutylbromide, deprotection reaction that removes Boc protecting group of the terminal primary amino group of PMPC is performed following the polymerization reaction.

10. The method according to claim 7, wherein the medium-molecular-weight compound is a single-stranded or double-stranded oligonucleotide having a molecular weight of 50 kDa or less.

11. The method according to claim 10, wherein the oligonucleotide is a DNA aptamer or an RNA aptamer.

12. The method according to claim 7, wherein the medium-molecular-weight compound is a peptide or a protein having a molecular weight of 50 kDa or less.

13. The method according to claim 7, wherein the medium-molecular-weight compound is a nanobody, a Fab fragment of an antibody, a peptide hormone, a chemokine, a cytokine, a linear or cyclic peptide having a function to bind to a specific protein.

14. The method according to claim 7, wherein the medium-molecular-weight compound and PMPC are chemically bound through a linker.

15. The method according to claim 14, wherein the linker and PMPC are bound by condensation reaction of an active ester and an amine, or condensation reaction of an amine and an intermediate formed by activating an ester or a carboxylic acid in a reaction system or outside a reaction system.

16. The method according to claim 15, wherein the active ester is N-hydroxysuccinimide ester.

17. The method according to claim 7, wherein the medium-molecular-weight compound has a functional group used for click reaction, and the functional group and PMPC are bound by click reaction.
